(19) United States
(12) Patent Application Publication
(10) Pub. No.: US 2022/0354483 A1
(43) Pub. Date: Nov. 10, 2022

(54) TISSUE ANCHORING DEVICE AND USE THEREOF

(71) Applicant: NANOVA BIOMATERIALS INC., Columbia, MO (US)

(72) Inventors: Richard Joseph Lebens, III, Columbia, MO (US); Richard Alexander White, Columbia, MO (US); Kevin Jerrel Loeppke, Columbia, MO (US)

(21) Appl. No.: 17/740,216

(22) Filed: May 9, 2022

Related U.S. Application Data
(60) Provisional application No. 63/186,473, filed on May 10, 2021.

(51) Int. Cl. A61B 17/04

(52) U.S. Cl. CPC A61B 17/0401; A61B 2017/0441; A61B 2017/0438

(57) ABSTRACT

A tissue anchor for use in holding a tissue to a bone is provided. The anchor includes a distal tip, a proximal end, and an elongated body extending from the distal tip to the proximal end along a longitudinal axis. The elongated body can include a cannulated tunnel having an opening at the proximal end. An engagement member can be disposed inside of the elongated body and configured to enable the loading of a first thread-like fixation element through the cannulated tunnel. The distal tip of the tissue anchor can include an opening dimensioned and configured to capture and retain a second thread-like fixation element.

### BACKGROUND

In the field of sports medicine has experienced a rapid growth with the growing and aging population. The number of tissue repair procedures, e.g., in the knee, hip, ankle, shoulder, elbow, and other tendon and ligament joints, has increased considerably. There is also a demand for improved technology for improved fixation, shorter operation time, and better operating complexity in tissue repair procedures.

More than half of tissue fixation procedures are in the shoulder. The most common shoulder injuries occur are rotator cuff and glenoid labral tears. Briefly, the shoulder joint is formed by the articulation of the head of the humorous and the glenoid cavity (glenohumeral joint). This synovial joint of the spheroidal (ball and socket) type is similar to the hip, however, the glenoid socket is shallower in comparison to the acetabulum of the hip. From this shallowness arises the inherent instability of the shoulder system. In order to overcome this deficiency in the glenoid socket is drastically deepened by a fibrocartilage rim (glenoid labrum). The shoulder socket's vast movement is primarily supported by 4 tendons connecting the humorous and forming a capsule of fused tendons. These tendons are known as the rotator cuff and include the Subscapularis, Supraspinatous, Infraspinatus, and Teres minor. When any of these tendons or ligaments are damaged, instability often follows.

Where shoulder instability is present a primary course of action is surgical repair. In this type of operations, tendon/ligaments are reconnected and brought back to the natural physiological position. Arthroscopic stabilization surgical technique has become the most popular and preferred method to repair the labrum and rotator cuff tendons. In general, this method involves the utilization of soft tissue fixation anchors. For example, the anchor is first secured in the bone of the humorous or glenoid. Then the torn tissue ligament or tendon, which has been captured with suture, is brought to the bone surface of the glenoid or humorous and attached to the anchor. Post-operation the tendon or cartilage will be fused to the surface of the bone over time.

Currently, available tissue anchors are plagued with many problems. Primary modes of failure include failure during insertion, anchor eyelet failure, suture failure, and loosening. For example, during insertion (into bone) the anchor may not be able to resist the torque required to fully drive into the preformed tunnel, which may be caused by techniques or material/design of the anchor. In early generation anchor systems eyelets were proximal at the head, so that the eyelets would need to endure both the loading during insertion as well as physiological loading. Also, the original design of tissue anchors distributed the majority of stress to the eyelet of the anchor during insertion.

Soft tissue anchors are now used in indications across the entire body with applications in the hip, ankle, foot, knee, and many more. In some of such anchors, a screw is used as the delivery mechanism with a proximal eyelet where suture can be attached. In some soft tissue anchors, the eyelet is positioned the distal end of the screw. This solved many of the problems associated with eyelet breaking and improved fixation. One problem remaining in the screw in type tissue anchor is the amount of space needed because the insertion device needs to be larger to resist torque forces during insertion. Most screw in anchors have an outer diameter of 4.5 mm or larger. For specific applications such as the glenohumeral repair such space is simply not available.

U.S. Pat. No. 5,601,557 describe a type of anchor referred to as a “push-in” anchor today. This type of anchor is tapped/punched into a pilot hole preformed in the bone. In this push-in operation barb-like features of the anchor deform as it enters the pilot hole. Once deployed the barb-like features provides added friction between the anchor and the bone mass, thereby providing the fixation of the tissue to the bone. This allows for simplified fixation, shorter surgery time, and smaller overall footprint and size of the anchor device.

U.S. Pat. No. 5,709,708 describes a knotless tissue anchor having a snag means for securing a suture attached to the anchor when the anchor is driven into a bone allowing the suture to create an interference fit holding the tissue in position.

The advances in the design of tissue anchor system have provided surgeons with more options and greater chance of success. However, the available anchoring devices still do not give surgeons sufficient flexibility. For instance, using the knotless “push-in” technique, once the anchor is installed in the bone, if the tissue fixation is still loose the tissue cannot be further manipulated without the addition of another anchor, which would induce a larger device footprint than desired. Additionally, knotless techniques for attaching tissue are cumbersome and complex, requiring extensive training and familiarity on the part of the surgeons to perform the operations.

### SUMMARY OF THE INVENTION

In one aspect, a tissue anchor for use in holding a tissue to a bone is provided. The tissue anchor includes: a distal tip; a proximal end; an elongated body extending from the distal tip to the proximal end along a longitudinal axis, the elongated body comprising at least one lumen; an engagement member disposed inside of the elongated body configured to enable the loading of a first thread-like fixation element through the at least one lumen; wherein the distal tip defines an opening dimensioned and configured to capture and retain a second thread-like fixation element.

In some embodiments, the at least one lumen comprises a cannulated tunnel defined axially in at least a portion of the elongated body and having an opening at the proximal end, the cannulated tunnel having a proximal end and a distal end, and wherein the engagement member is disposed at the distal end of the cannulated tunnel.

In some embodiments, the engagement member has a distal end such that the loading of the first thread-like fixation results in the following configuration: the first thread-like fixation element enters the cannulated tunnel from the proximal end, wraps around the distal end of the engagement member, and exits the cannulated tunnel from the proximal end.

In some embodiments, the tissue anchor further includes the first thread-like fixation element preloaded with the engagement member and accommodated within in the cannulated tunnel from the proximal end.

In some embodiments, the elongated body includes one or more connected segments along its longitudinal axis, each of the segment having a cross-section profile with decreasing cross-section area in the direction from the proximal end to the distal tip.

In some embodiments, the elongated body can be deflectable along its longitudinal axis.

In some embodiments, the distal tip has a generally conical shape or outer profile.

In some embodiments, the opening has a generally “U” -shaped cross section viewed from a direction perpendicular to the longitudinal axis.

In some embodiments, the engagement member and the elongated body form two channels on each side of the longitudinal axis, the two channels being parallel to each other. The channels can be used to accommodate a first thread-like fixation element as a loop. The two channels can be each parallel to the direction of the opening of the distal tip. Alternatively, the two channels are each non-parallel to the direction of the opening of the distal tip, e.g., perpendicular to the opening of the distal tip.

In some embodiments, the opening includes two opposing side walls and a bottom (or valley) and at least one hindrance element extending from a first side wall of the opening and protruding toward the valley and against the second, opposing side wall, the configuration of the hindrance element allows capture of the second thread-like fixation element into the opening and hinders the captured second thread-like fixation element from escaping from the opening.

In some embodiments, the at least one hindrance element comprises a plurality of hindrance elements disposed on opposing side walls of the opening. The hindrance element can be a single hindrance element, and the opening of the distal tip is without any other hindrance element.

In another aspect, the present disclosure provides a tissue anchor for use in holding a tissue to a bone, which includes: a distal tip; a proximal end; an elongated body extending from the distal tip to the proximal end along a longitudinal axis, the elongated body comprising at least one lumen; and an engagement member disposed inside of the elongated body configured to enable the loading of a first thread-like fixation element inside the cannulated tunnel through the at least one lumen. In some embodiments, the at least one lumen includes a cannulated tunnel defined axially in at least a portion of the elongated body and having an opening at the proximal end, the cannulated tunnel having a proximal end and a distal end, and wherein the engagement member is disposed at the distal end of the cannulated tunnel. The distal tip can define an opening dimensioned and configured to capture and retain a second thread-like fixation element.

In a further aspect, the present disclosure provides a tissue anchor for use in holding a tissue to a bone, which includes: a distal tip; a proximal end; an elongated body extending from the distal tip to the proximal end, the elongated body including at least one lumen; wherein the distal tip defines an opening configured to capture or retain a second thread-like fixation element, the opening including two opposing side walls and a valley, and a single hindrance element extending from a first side wall of the opening and protruding toward the valley and against the second, opposing side wall, the configuration of the hindrance element allows capture of the second thread-like fixation element into the opening and hinders the captured second thread-like fixation element from escaping from the opening; the opening being without other hindrance element; the opening not including any other hindrance element. The hindrance element can taper in thickness toward its tip. In some embodiments, the tissue anchor further includes an engagement element disposed in the elongated body or formed on the elongated body, the engagement element configured to enable the loading of a first thread-like fixation element through the at least one lumen. The elongated body can define a cannulated tunnel which has an opening at a proximal end; wherein the engagement element includes a barrier member disposed inside of the elongated body, wherein the engagement member has a distal end such that the loading of the first thread-like fixation results in the following configuration: the first thread-like fixation element enters the cannulated tunnel from the proximal end, wraps around the distal end of the engagement member, and exits the cannulated tunnel from the proximal end.

### BRIEF DESCRIPTION OF THE DRAWINGS

FIG. 1A is a perspective view of a tissue anchor device according to an embodiment of the present invention.

FIG. 1B is a proximal end view of the tissue anchor device shown in FIG. 1A.

FIG. 1C is a perspective sectional view of a distal portion of the tissue anchor device shown in FIG. 1A.

FIG. 1D is a sectional view of a distal portion of the tissue anchor device shown in FIG. 1A.

FIG. 2A is a perspective view of a tissue anchor device according to an embodiment of the present invention. FIG. 2B is a proximal end view of the tissue anchor device shown in FIG. 2A.

FIG. 2C is a perspective view of a tissue anchor device according to an embodiment of the present invention. FIG. 2D is a proximal end view of the tissue anchor device shown in FIG. 2C.

FIG. 2E is a perspective view of a tissue anchor device according to an embodiment of the present invention. FIG. 2F is a proximal end view of the tissue anchor device shown in FIG. 2E.

FIG. 2G is a perspective view of a tissue anchor device according to an embodiment of the present invention. FIG. 2H is a proximal end view of the tissue anchor device shown in FIG. 2G.

FIG. 3A is a perspective sectional view of a tissue anchor device according to an embodiment of the present invention loaded with a first suture and a second suture. FIG. 3B is a perspective view of the tissue anchor device shown in FIG. 3A.

FIG. 4A is a perspective sectional view of a tissue anchor device according to an embodiment of the present invention loaded with a first suture and a second suture. FIG. 4B is a perspective view of the tissue anchor device shown in FIG. 4A.

FIG. 5A is a side view of a tissue anchor device of the present invention with a second suture element captured at its distal tip opening. FIG. 5B is a close-up sectional view of the tissue anchor shown in FIG. 5A along its longitudinal axis, showing a first suture element preloaded on an engagement member and concealed in the annulated tunnel of the anchor device. FIG. 5C is a close-up sectional view of a tissue anchor along its longitudinal axis, showing multiple suture elements captured at its distal tip opening, and a first suture element preloaded in the cannulated tunnel of the anchor device.

FIG. 6A is a side view of a distal portion (including the distal tip and opening) of a tissue anchor device according to one embodiment of the present invention. FIG. 6B is an interior sectional view of a distal portion of the tissue anchor device shown in FIG. 6A. FIG. 6C is a side view of a distal portion (including the distal tip and opening) of a tissue anchor device according to another embodiment of the present invention.

FIG. 7A is a perspective view of a distal tip opening of a tissue anchor device according to another embodiment of the present invention. FIG. 7B is a perspective view of the distal tip opening of the tissue anchor device shown in FIG. 7A with suture element captured in the distal tip opening. FIG. 7C is a perspective view of a distal tip opening of a tissue anchor device according to another embodiment of the present invention. FIG. 7D is a perspective view of the distal tip opening of the tissue anchor device shown in FIG. 7C with suture element captured in the distal tip opening.

FIGS. 8A and 8B are schematic depictions of a tissue anchor device of the present invention as being used to secure a tissue to the bone via the tissue anchor device capturing a suture loop in the distal tip opening and being inserted into a preformed bone hole.

### DETAILED DESCRIPTION OF THE INVENTION

Embodiments the invention will described more fully hereinafter, with accompanying references to the figures in which embodiments of the invention are shown, where like reference numbers denote like features.

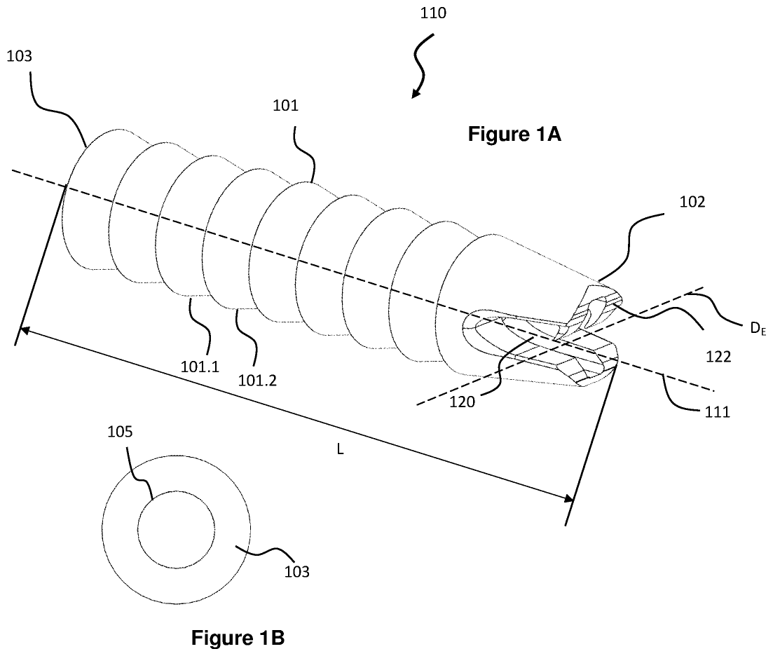

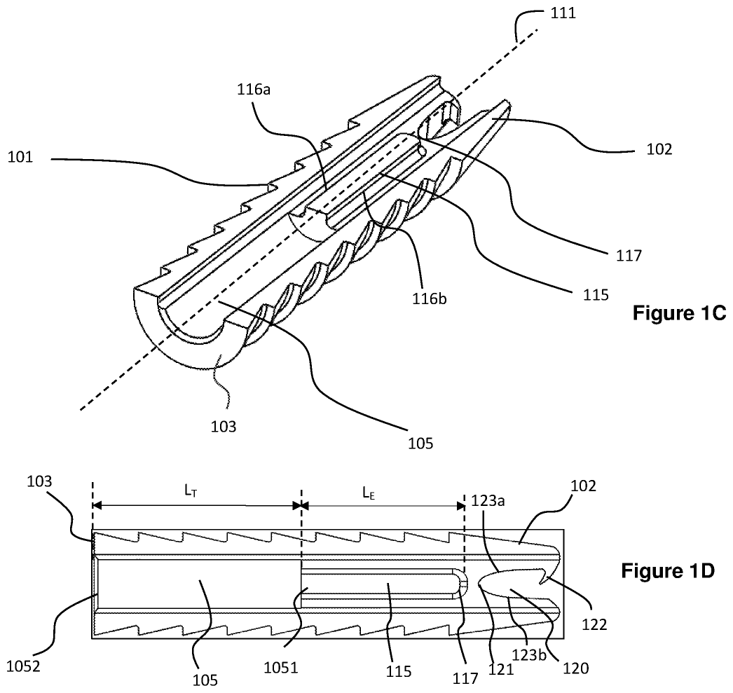

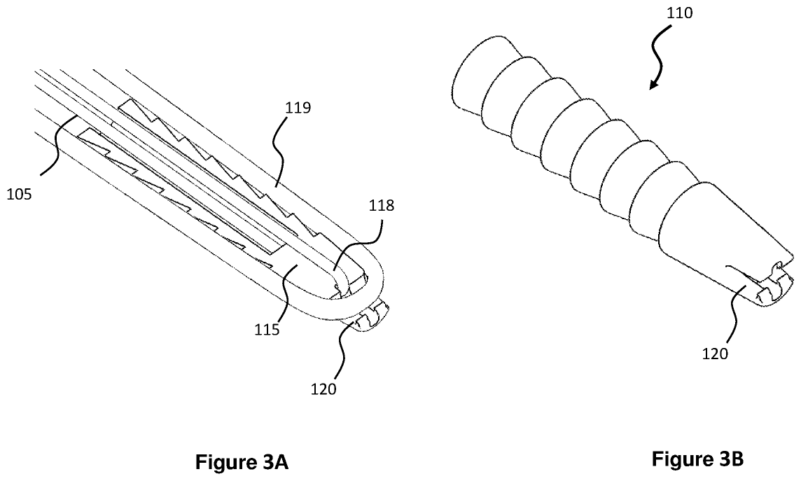

Referring to FIGS. 1A-1D, a tissue anchoring device (or tissue anchor) 110 includes a distal tip 102, a proximal end 103, and an elongated body 101 extending from the distal tip to the proximal end along a longitudinal axis 111. A cannulated tunnel 105 is defined axially in the elongated body, has a distal end 1051 and an opening at a proximal end 1052 (which is also the proximal end 103 of the tissue anchor). An engagement member 115 (having a distal end 117 and an axial length of L_{E}) is disposed at the distal end 1051 of the cannulated tunnel 105 and inside of the elongated body, and configured to enable the loading of a first thread-like fixation element (or suture element as referred herein) inside the cannulated tunnel 105 (the loading of the first suture element which will be further illustrated and described in connection with FIGS. 3A-3B, 4A-4B, 5A-5C). The engagement member 115 and the elongated body form two channels 116a and 116b on each side of the longitudinal axis 111, the two channels being parallel to each other and each are in open/fluid communication with the tunnel. The channels 116a/116b and the tunnel 105 can be considered lumen of the elongated body 101. The distal tip 102 defines an opening 120 (which is also referred as the “distal eyelet”, “open eyelet” or “capturing eyelet”) dimensioned and configured to capture and retain a second thread-like fixation element (or suture element as referred herein). The total length of the anchor device is denoted L.

In alternative embodiments, the tissue anchor can be without the cannulated tunnel 105 as shown in FIGS. 1A-1D, and the channels along the engagement member may run the length toward the proximal end of the tissue anchor. Although it is shown that the cannulated tunnel is void throughout in the drawings, it is understood that it be interrupted by structures such as reinforcing bars as long as the fixation element (suture) can run through the tunnel freely.

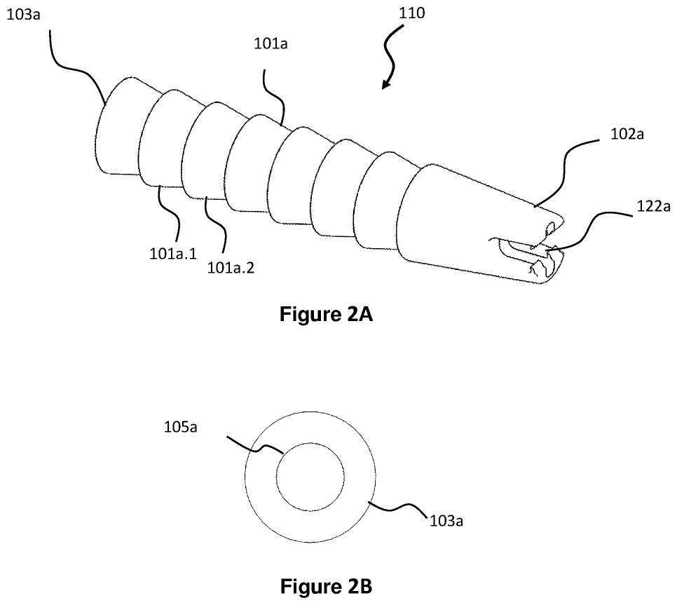

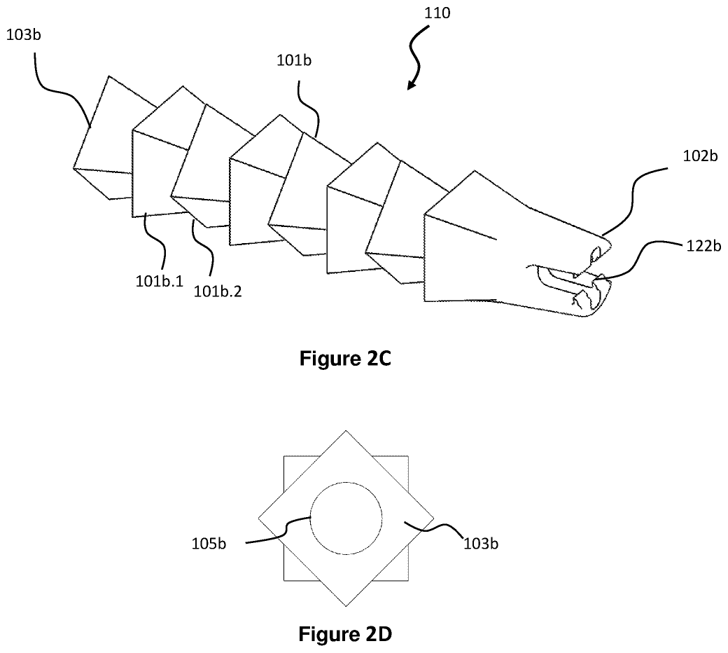

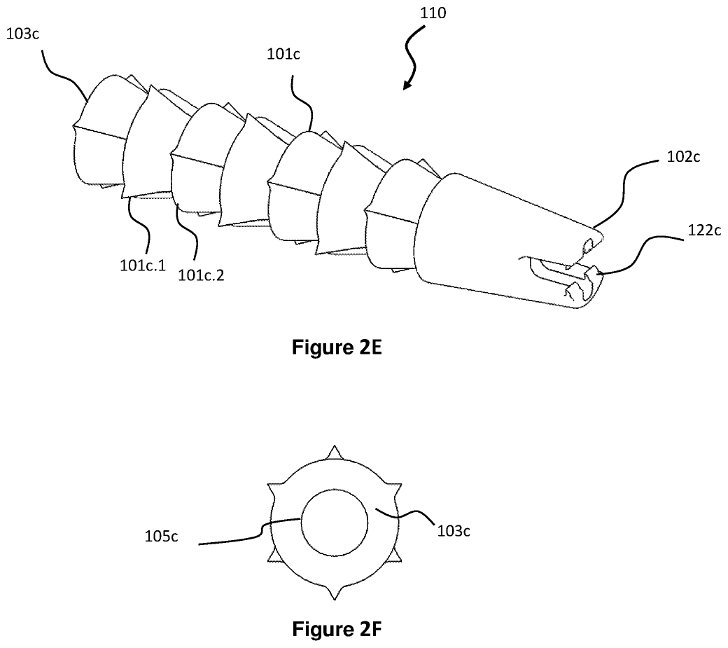

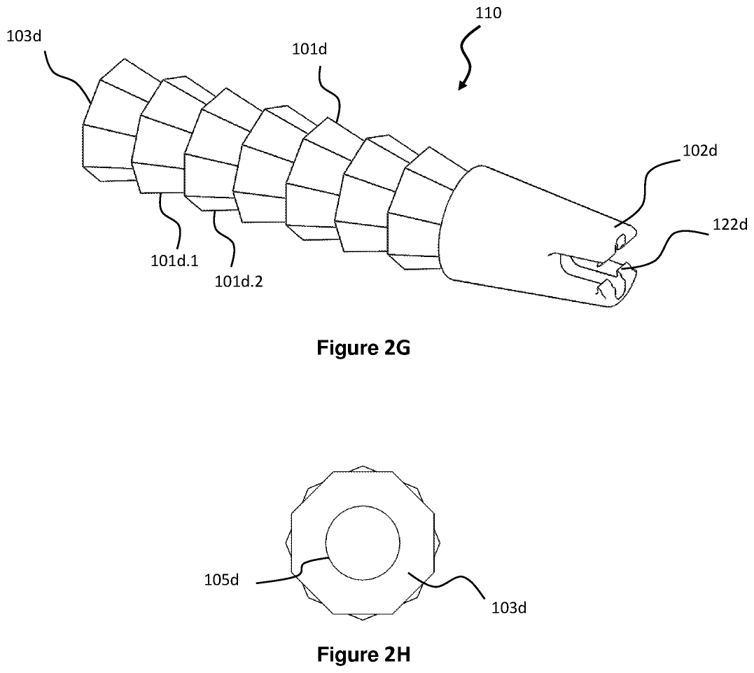

As shown in FIGS. 1A, 1C and 1D, the opening 120 has a generally decreasing profile from top to bottom, e.g., having generally “U”-shaped cross section viewed from a direction D_{E} perpendicular to the longitudinal axis. It has two opposing side walls 123a, 123b and a bottom 121 (or valley), shown in FIG. 1D. The opening 120 further comprises at least one hindrance element 122 extending from a first side wall 123a of the opening and protruding toward the valley 121 and against the second side wall 123b. The girth/width/thickness of the hindrance element can taper from its base to its tip. The configuration of the hindrance element allows capture of the second suture element into the opening and hinders the captured second suture element from escaping from the opening. As shown in these figures, the opening 120 has only one hindrance element which can extend into the opening to about half of the width of the opening. In other embodiments, as shown in FIGS. 2A, 2C, 2E, 2G, for example, the opening 120 can have multiple hindrance elements extending from opposing walls. These hindrance element(s) may extend from the distal end of the opening as shown, or from another position as desired. The hindrance element(s) may be flexible, and/or has a bias in terms of bendability—it can be easily bent inward toward the valley of the opening, but more difficult to bend outward. This can allow easy capture of sutures and retraining the captured sutures from escaping.

FIGS. 2A-2H show other embodiments of tissue anchoring devices having a distal tip (102a; 102b; 102c; 102d), a proximal end (103a; 103b; 103c; 103d), and elongated body (101a; 101b; 101c; 101d) extending between the distal tip to the proximal end.

The exterior of the anchor can include friction-enhancing features that allow it to be pushed through a bone hole in one direction but cannot be retrieved easily from the reverse direction. As shown in FIG. 1A, the elongated body 101 can include a plurality of segments (e.g., 101.1, 101.2 as shown) each having a cross-section profile with decreasing cross-section area in the direction from the proximal end to the distal tip. When the anchor is made from a deformable/resilient material, the outer diameter of the anchor can be slightly larger than the diameter of the hole. The combination of the multi-segment exterior feature with the hollow interior allows the anchor to be securely fixated in the bone hole after being pushed in place.

While the segments generally have circular cross-sectional shapes as shown in FIG. 1A, FIGS. 2A-2H show other embodiments of tissue anchoring devices including segments (101a.1, 101a.2; 101b.1, 101b.2; 101c.1, 101c.2; 101d.1, 101d.2) having varying cross sectional shapes, for example, circular, rectangular, circular plus barbes/ridges, and octagonal, and other polygonal or irregular shapes as desired. When the cross-sectional shapes are not circular, the edges or ridges of adjacently connected segments can be distributed in a staggered manner (or alternating in orientation), as shown in FIGS. 2C-2H.

As shown in FIGS. 1A-1D and 2A-2H, the distal tip generally has a conical shape with tapering end. Together with the opening 120, this configuration allows easy insertion into the preformed bone hole and locking of suture to the anchor device.

As will be further described herein, embodiments of the tissue anchor of the present invention can be used in both knot-tying and knotless applications. The “push-in” type configuration has a small footprint as to not interfere with the physiological anatomical movements and biological response.

In some embodiments, the total length of the anchor L (including the distal tip) can be at least twice as great as the diameter or width of the anchor. Although shown the cannulated tunnel as generally cylindrical, the tunnel can be in any cross-sectional shape, such as circular, rectangular, other polygonal shape, or irregular shape. The individual parts of the tissue anchor (e.g., the distal tip, the connected segments of the main body) can be but need not be made of a singular body. For example, the distal tip and elongated body may be made of one piece or different materials.

In some embodiments, the distance between the proximal end of the elongated body and the distal end of the engagement member (i.e., L_{T}+L_{E} shown in FIG. 1D) can be at least 50% of the total length of the tissue anchor L.

In the embodiments shown and described herein, the main or elongated body 101 (or 101a, 101b, 101c, 101d), the distal tip 102 (or 102a, 102b, 102c, 102d), and the other components of the tissue anchor assembly are preferably formed of biocompatible and/or bioabsorbable materials, including but not limited to metals or metallic materials such as stainless steel, titanium, nickel, nickel-titanium alloy (i.e., Nitinol) or other alloys, plastics or other polymeric materials, biocompatible or bioabsorbable (e.g., PGA, PLA, PLG and other lactide-glycolide polymers and copolymers) medical grade materials conventionally used for tissue anchors, sutures, implants, and similar devices. Tissue anchor assembly embodiments can be formed of combinations of these materials. In some embodiments, the tissue anchor (or parts thereof) can be made from elastic/deformable materials. In some embodiments, the tissue anchor can be deflectable along its longitudinal axis.

In various embodiments of the tissue anchor device, the proximal end of the cannulated tunnel can include features, for example, specifically gauged holes, to engage an external driver, or geometry to allow a driver rod to be inserted and manipulate the anchor in desired mode of motion, e.g., linear axial movement, rotating about the longitudinal axis, or otherwise. The driver rod can be inserted into the cannulated tunnel. This can reduce the risk of failure during insertion of the anchor into the preformed bone hole.

While it has been described that the tissue anchor device can include exterior features to facilitate push-in installation (e.g., those specific geometries of the connected segments, as well as the ribs or barb features that allow one-way easy insertion of the anchor device with its distal tip toward the bone hole), the exterior of the elongated body can also have threads that allow “screw-in” installation.

One example method of inserting the anchor into bone/tissue is by means of first forming a bone hole with a drill or punch, which is undersized as compared to the outer diameter of the tissue anchoring device. The bone hole can be made slightly deeper than the overall length of the anchor. This is done with preset length provided with the anchor set. Sutures from tissue are captured from with the distal tip and secured by the teeth like exterior features of the anchor as shown and described herein.

Additionally, the conical design inhibits the chance of non-co-axial insertion. Prior to driving the anchor device into the bone the tissue can be secured and a better anatomical position can be set. Following this the anchor can be driven into the bone hole. The tissue anchoring device can be driven flush to the bone surface, or be driven under the bone surface if desired. This is a reason a deeper pre-drilled hole is made. Following fixation the surgeon can determine the fixation strength and access the repair. If necessary, the preloaded suture may now be used to further increase the fixation strength, used on another tissue (ligament or tendon), or removed completely.

FIGS. 3A and 3B show perspective views of an embodiment of the present invention. FIG. 3A shows a section view where it has been cut along a plane in the middle of the anchor running axially along the anchor, and FIG. 3B shows the exterior view of the same embodiment and orientation. Suture element 118 is loaded on the engagement member 115 and concealed in the tunnel 105 within the length of the anchor. In use suture element 118 may be preloaded on the anchor and suture 119 may be captured by the open eyelet 120. As shown, the first suture element 118 and the second suture element 119 are perpendicular to each other when viewed down the main axis of the anchor. This may reduce friction and increase sliding and versatility of the anchor once inserted in bone.

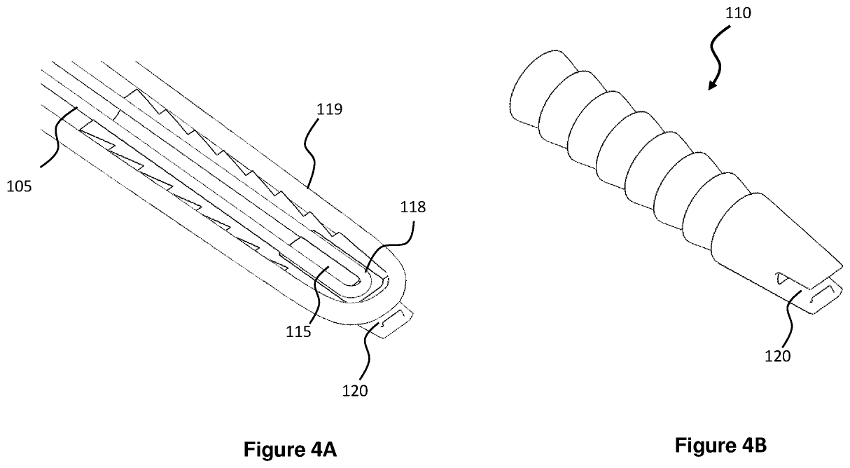

FIGS. 4A and 4B show perspective views of a tissue anchor of another embodiment of the present invention. FIG. 4A shows a section view where the anchor has been cut along a plane in the middle of the anchor running axially along the anchor, and FIG. 4B shows the exterior view of the anchor of the same embodiment and orientation. Suture element 118 is loaded on the engagement member 115 and concealed in the tunnel 105 within the length of the anchor. In use suture 118 may be preloaded on the anchor and suture 119 may be captured by the open eyelet 120. The first suture element 118 and the second suture element 110 are parallel to each other when viewed down the main axis of the anchor.

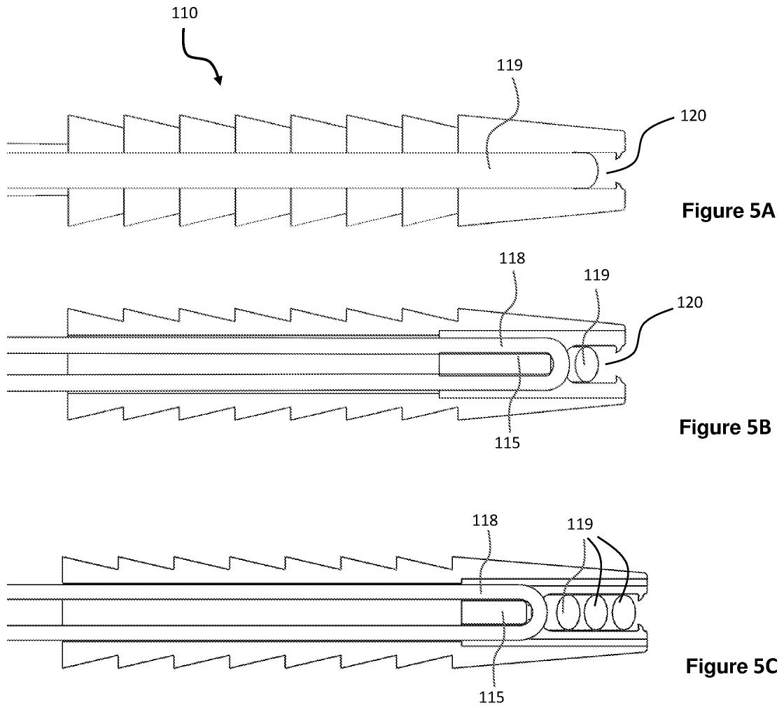

FIG. 5A shows an embodiment of anchor 110 and suture 119 that has been captured by open eyelet 120. FIG. 5B shows a section view of the embodiment in FIG. 5A cut on the plane in the middle of the anchor perpendicular to the viewer. Suture 118 which may be preloaded on the engagement member 115 in the anchor can be seen in this section view. It can be more clearly seen by these two figures that sutures 118 and 119 are perpendicular to each other. FIG. 5C is a section view similar to FIG. 5B, but displays an embodiment where the open eyelet 120 has been lengthened to be capable of capturing more sutures(three are shown in this figure).

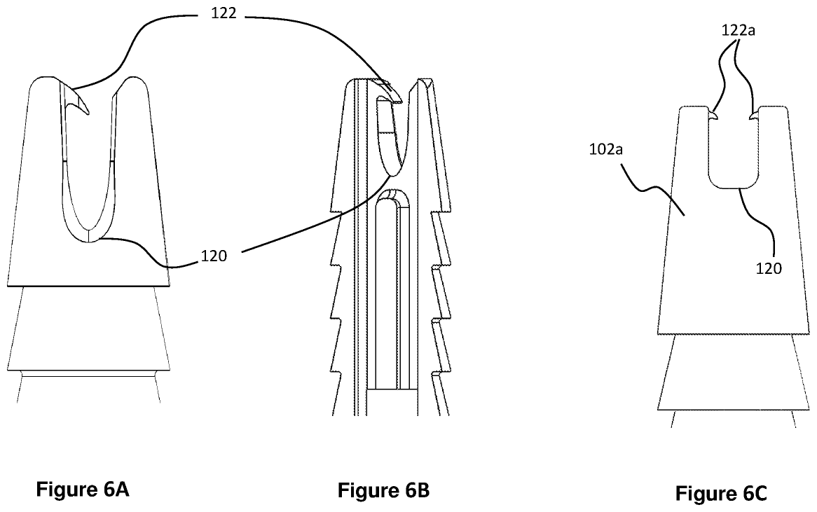

FIGS. 6A and 6B are close-up views of a distal portion of a tissue anchor shown in FIG. 1A/1C/1D, showing the open eyelet 120 and the single hindrance element 122. An external suture element would enter through opening seen at the top of the eyelet. FIG. 6C is a close-up view of a distal portion of a tissue anchor according to an alternative embodiment (as seen in FIG. 2A), where hindrance elements are extending from opposing side walls of the opening toward the center of the opening. In some embodiments, the hindrance element can extend up to the central axis or beyond the central axis of the generally “U”-shaped cross section.

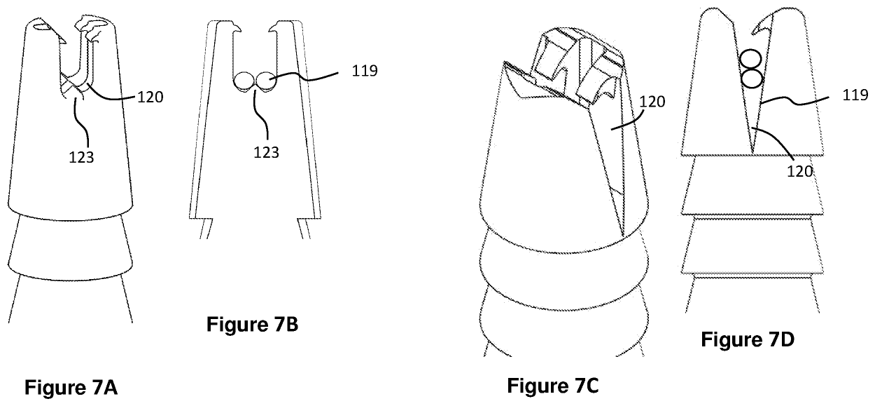

FIG. 7A shows a close-up view of the distal portion of a tissue anchor of another embodiment, with a section view shown in FIG. 7B. Open eyelet 120 can be seen for capturing external sutures. This embodiment shows a center bump 123 on the bottom of the open eyelet 120 which may increase separation of sutures captured 119. This center bump may include sharp or rounded edges, and may increase the maximum force the anchor can resist before failure.

In another aspect of the present invention, a method for the attachment of tissue to a bone using the tissue anchor described herein, is provided. In the method, the opening of the distal tip of the tissue anchor is used to capture a second suture element which forms a continuous loop through the tissue. Then, the tissue anchor is installed into bone with the captured second suture element, thereby securing the second suture element to the bone. Before the procedure, a hole can be made on the desired location on the bone using a drill or other suitable tools.

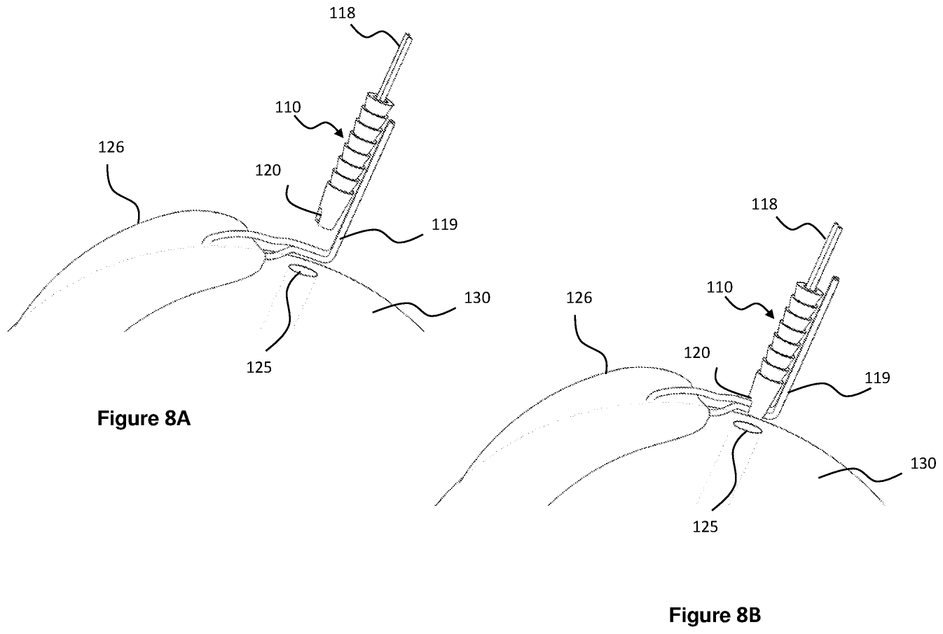

FIGS. 8A and 8B depict schematically attaching tissue 126 to bone 130. FIG. 8A shows the various components before both strands of suture 119 (which forms a continuous loop after going through the tissue to be secured) are captured by open eyelet 120, and FIG. 8B shows the various components after suture 119 has been captured by open eyelet 120. Suture 118 can also be seen which may have been pre-loaded in the anchor. The loading of the first suture element results in the following configuration: the first suture element enters the cannulated tunnel from the proximal end, wraps around the distal end of the engagement member, and exits the cannulated tunnel from the proximal end. As the engagement member is disposed inside of the anchor, and the portion of the first suture element running inside the tunnel of the anchor is concealed, one can only see the ends of the first suture exposed out of the proximal end of the anchor.

The anchor may then be inserted in the preformed bone hole 125 after suture 119 has been captured to provide fixation of tissue 126. Suture 119 may be held in place in the bone hole by anchor 110 by friction between the bone mass and the exterior anchor surface once it is inserted into the bone hole. At full insertion, the proximal end of the anchor may be flush with the surrounding bone. If the fixation with suture 119 is not satisfactory, suture 118 may be used to provide additional fixation or security. If no additional fixation is desired, suture 118 may be pulled out of the anchor if desired (by pulling one strand of the suture) once the anchor has been inserted, and discarded. The insertion of the anchor 110 into the bone hole 125 may be accomplished by the assistance of a driver shaft inserted into the cannulated tunnel of the anchor. The driver shaft may be hollow or has other features that allow it to not interfere with the preloaded suture 118 in the tunnel.

Although the present invention has been described in relation to particular embodiments thereof, many other variations and modifications and other uses will become apparent to those skilled in the art. It is therefore contemplated that the appended claims will cover any such modifications or embodiments that fall within the scope of the invention. The present invention is to not be limited by the specific disclosure herein.

1. A tissue anchor for use in holding a tissue to a bone, comprising:
a distal tip;
a proximal end;
an elongated body extending from the distal tip to the proximal end along a longitudinal axis, the elongated body comprising at least one lumen;
an engagement member disposed inside of the elongated body configured to enable the loading of a first thread-like fixation element through the at least one lumen; and
wherein the distal tip defines an opening dimensioned and configured to capture and retain a second thread-like fixation element.

2. The tissue anchor of claim 1, wherein the at least one lumen comprises a cannulated tunnel defined axially in at least a portion of the elongated body and having an opening at the proximal end, the cannulated tunnel having a proximal end and a distal end, and wherein the engagement member is disposed at the distal end of the cannulated tunnel.

3. The tissue anchor of claim 2, wherein the engagement member has a distal end such that the loading of the first thread-like fixation results in the following configuration: the first thread-like fixation element enters the cannulated tunnel from the proximal end, wraps around the distal end of the engagement member, and exits the cannulated tunnel from the proximal end.

4. The tissue anchor of any of claims 2-3, further comprising the first thread-like fixation element preloaded with the engagement member and accommodated within in the cannulated tunnel from the proximal end.

5. The tissue anchor of any of claims 1-4, where the elongated body comprises one or more connected segments, each of the segment having a cross-section profile with decreasing cross-section area in the direction from the proximal end to the distal tip.

6. The tissue anchor of any of claims 1-5, wherein the elongated body is deflectable along its longitudinal axis.

7. The tissue anchor of any of claims 1-6, wherein the distal tip is generally conical in shape.

8. The tissue anchor of any of claims 1-7, wherein the opening has a generally “U” -shaped cross section viewed from a direction perpendicular to the longitudinal axis.

9. The tissue anchor of any of claims 1-8, wherein the engagement member and the elongated body form two channels on each side of the longitudinal axis, the two channels being parallel to each other.

10. The tissue anchor of claim 9, wherein the two channels are each parallel to the direction of the opening of the distal tip.

11. The tissue anchor of claim 10, wherein the two channels are each non-parallel to the direction of the opening of the distal tip.

12. The tissue anchor of any of claims 1-11, wherein the opening comprises two opposing side walls and a bottom (or valley) and at least one hindrance element extending from a first side wall of the opening and protruding toward the valley and against the second, opposing side wall, the configuration of the hindrance element allows capture of the second thread-like fixation element into the opening and hinders the captured second thread-like fixation element from escaping from the opening.

13. The tissue anchor of claim 12, wherein the at least one hindrance element comprises a plurality of hindrance elements disposed on opposing side walls of the opening.

14. The tissue anchor of claim 12, wherein the at least one hindrance element consists of a single hindrance element, and the opening of the distal tip is without any other hindrance element.

15. A tissue anchor for use in holding a tissue to a bone, comprising:
a distal tip;
a proximal end;
an elongated body extending from the distal tip to the proximal end along a longitudinal axis, the elongated body comprising at least one lumen; and
an engagement member disposed inside of the elongated body configured to enable the loading of a first thread-like fixation element inside the cannulated tunnel through the at least one lumen.

16. The tissue anchor of claim 15, wherein the at least one lumen comprises a cannulated tunnel defined axially in at least a portion of the elongated body and having an opening at the proximal end, the cannulated tunnel having a proximal end and a distal end, and wherein the engagement member is disposed at the distal end of the cannulated tunnel.

17. The tissue anchor of claim 16, wherein the distal tip defines an opening dimensioned and configured to capture and retain a second thread-like fixation element.

18. A tissue anchor for use in holding a tissue to a bone, comprising:
a distal tip;
a proximal end;
an elongated body extending from the distal tip to the proximal end, the elongated body comprising at least one lumen;
wherein the distal tip defines an opening configured to capture or retain a second thread-like fixation element, the opening having generally decreasing profile in cross section and comprising two opposing side walls and a valley; and
wherein the opening further comprises a single hindrance element extending from a first side wall of the opening and protruding toward the valley and against the second, opposing side wall, the configuration of the hindrance element allows capture of the second thread-like fixation element into the opening and hinders the captured second thread-like fixation element from escaping from the opening; the opening being without other hindrance element; the opening not including any other hindrance element.

19. The tissue anchor of claim 18, further comprising:
an engagement element disposed in the elongated body or formed on the elongated body, the engagement element configured to enable the loading of a first thread-like fixation element through the at least one lumen.

20. The tissue anchor of claim 19, wherein the elongated body defines a cannulated tunnel which has an opening at a proximal end, and wherein the engagement element comprises a barrier member disposed inside of the elongated body, and the engagement member has a distal end such that the loading of the first thread-like fixation results in the following configuration: the first thread-like fixation element enters the cannulated tunnel from the proximal end, wraps around the distal end of the engagement member, and exits the cannulated tunnel from the proximal end.
